(19) United States
(12) Patent Application Publication
(10) Pub. No.: US 2026/0053732 A1
(43) Pub. Date: Feb. 26, 2026

(54) DEVICE FOR MAINTAINING METAL HOMEOSTASIS, AND USES THEREOF

(71) Applicants: MEXBRAIN, Villeurbanne (FR); UNIVERSITE CLAUDE BERNARD LYON 1, Villeurbanne (FR); CENTRE NATIONAL DE LA RECHERCHE SCIENTIFIQUE - CNRS -, Paris (FR)

(72) Inventors: François LUX, Lyon (FR); Olivier TILLEMENT, Fontaines Saint-Martin (FR); Yannick CREMILLEUX, Merignac (FR); Thomas BRICHART, Lyon (FR); Sébastien GROYER, Paris (FR)

(21) Appl. No.: 18/999,447

(22) Filed: Dec. 23, 2024

Related U.S. Application Data
(63) Continuation of application No. 16/955,923, now abandoned.

(51) Int. Cl. A61K 9/00; A61K 9/51; A61K 31/395; A61M 1/16; B82Y 5/00; B82Y 30/00; B82Y 40/00

(52) U.S. Cl. CPC A61K 9/0024; A61K 9/5146; A61K 31/395; A61M 1/1654; B82Y 5/00; B82Y 30/00; B82Y 40/00

(57) ABSTRACT

A method for extracting metal cations from a biological fluid of a subject in need thereof employs dialyzing the subject with a dialysis device. The dialysis device includes a porous dialysis membrane, and a reservoir containing a perfusion fluid. At least one chelator which is either grafted on polymers or grafted on nanoparticles is present in the perfusion fluid. The cut-off threshold of the porous membrane is lower than the mass of the polymer or the nanoparticles to which the chelator is grafted. Metal cations from a biological fluid of a subject are extracted by complexing to the chelator during dialysis.

### CROSS REFERENCE TO RELATED APPLICATION

The present application is a continuation application of U.S. Ser. No. 16/955,923 filed on Jun. 19, 2020, which claims priority to PCT/FR2018/053528 filed on Dec. 21, 2018, which claims priority to French Patent Application 1763090, filed on Dec. 22, 2017.

### TECHNICAL FIELD

The present invention relates to the field of medical devices, more particularly devices for extracting metals from the body. These devices can be used, for example, to prevent and/or treat pathologies linked to dysregulation of metal homeostasis in the body, for example neurological diseases.

### BACKGROUND

The potentially deleterious effect of metals in the body, particularly at the neurological level, is highlighted by an increasing number of scientific studies (C. Marchetti et al., Biometals, 2014). Chelation therapy aimed at reducing the concentration of metal ions has already been used for many years in cases of acute metal poisoning. A number of chelators are already accepted in humans, each associated with a particular group of metals (G. Crisponi et al., Coordination Chemistry Reviews, 2015). Chelation therapy has also been shown to be an indispensable tool in the treatment of transfused patients with β-thalassemia. Patients who are transfused many times suffer from iron accumulation in the body. These iron deposits are regulated by intravenous or oral administration of iron chelators such as desferrioxamine, deferiprone or deferasirox (P. V. Bernhardt et al., Dalton Trans, 2007). Chelation therapy with D-penicillamine and trientine (oral) is also currently used to extract copper cations and treat Wilson's disease, resulting from a genetic abnormality affecting a copper transporter: ATP7B. This abnormality leads to copper overloads with an increase in copper circulating in the blood, resulting in deposits in organs, mainly the liver and brain (M. L. Schilsky, Clin. Liver. Dis., 2017). Chelation therapy has good efficacy in the case of presymptomatic treatment but less efficacy in the case of hepatic or neurological damage (M. Wiggelinkhuizen et al., Aliment Pharmacol., 2009), probably due to difficulty in reaching the target area and low specificity.

Unfortunately, chelation therapy is also misused intravenously to treat many other conditions (autism, claudication, etc.) without prior medical validation.

These misguided uses may have led to heavy side effects and even in the most tragic cases to the death of patients due to excessive dysregulation within the body of essential metal homeostasis (G. Crisponi et al., Coordination Chemistry Reviews, 2015).

A growing number of scientific studies highlight the important role that metals, particularly iron, but also copper, zinc, manganese and even aluminum, may play in many neurological disorders (E. J. McAllum et al., J. Mol. Neurosci., 2016). This is naturally the case for iron is overload neurodegeneration, which a rare disease associated with a genetic abnormality linked to iron accumulation in certain areas of the brain and which currently only benefits from palliative treatments (S. Wiethoff et al., Handb. Clin. Neurol., 2017). In addition, many studies have shown that iron tends to accumulate in the brain with age (J. Acosta-Cabronero et al., Journal of Neuroscience, 2016). Iron plays a crucial role in many brain functions such as mitochondrial respiration, myelin and neurotransmitter synthesis and metabolism (A. A. Belaidi et al., Journal of Neurochemistry, 2016). In the brain, iron is predominantly localized in the substantia nigra pars compacta and in the central grey nuclei with levels comparable to those in the liver. With age, iron tends to accumulate in certain regions of the brain where it is found predominantly associated with ferritin and neuromelanin. The areas where iron levels are most likely to increase are substantia nigra, putamen, globus pallidus, the caudate nucleus or the cortex, each of which is associated with different neurodegenerative disorders (D. J. Hare et al., Nat. Rev. Neurol., 2015). Several neurological diseases such as Alzheimer's, Parkinson's and Huntington's disease are accompanied by increased iron levels in specific areas leading to cell damage and oxidative stress (A. A. Belaidi et al., Journal of Neurochemistry, 2016). In Parkinson's disease, an increase in the amount of iron in substantia nigra, the region of the brain that is susceptible to Parkinson's disease degeneration, has been observed. The increase in iron levels is specific to substantia nigra and does not occur in other regions not affected by the disease. This increase in iron levels can lead to damage following the Fenton reaction and it is established that oxidative damage is one of the features of neurodegenerative diseases (S. Ayton et al., Biomed. Res. Int., 2014). Alzheimer's disease is also characterized by disturbances in the amounts of metals in the brain but associated with other brain regions and other proteins. Indeed, it appears that in this case an increase in iron levels and a decrease in copper levels are observed (S. F. Graham et al., J. Alzheimers Dis., 2014). Huntington's disease is another neurodegenerative disease involving movement disorders, cognitive decline and psychiatric problems. In this pathology, many markers of oxidative stress are observed in the brain, which may be related to dysregulation of iron homeostasis (S. J. A. van den Bogaard et al., International Review of Neurobiology, 2013). The increase in iron levels in several brain regions (putamen, caudate nucleus and pallidum) has been that of validated by several MRI studies, including Bartzorkis and co-workers (G. Bartzorkis et al., Archives of Neurology, 1999).

This abundant evidence of the role of iron homeostasis dysregulation in many neurodegenerative disorders has led scientists to study the impact of chelation therapy on these pathologies (Table 1). For example, deferiprone (used for the treatment of iron deposits occurring during transfusions for B-thalassemia) was used in a phase II clinical trial (DeferipronPD, NCT01539837) which included 22 patients (A. Martin-Bastida et al., Scientific Reports, 2017). In this clinical trial, treatment lasted 6 months and was well tolerated by patients. A decrease in iron levels was observed in the dentate and caudate nuclei. The reduction of iron levels in substantia nigra was observed in only 3 patients. No changes in iron levels in globus pallidus and putamen were observed. In this trial, a trend towards improvement in motor scores and quality of life was shown but was not statistically significant. Another trial using deferiprone was conducted (Fair-Park I) by another team which showed a decrease in iron levels in the substantia nigra and an improvement in motor scores but also without reaching statistical significance (G. Grolez et al., BMC Neurology, 2015). Due to the encouraging results of the Fair-Park I trial, a Fair-Park II trial began in 2016 (Table 1). In view of the encouraging results of the Parkinson's trials, deferiprone has recently been proposed for a clinical trial in Alzheimer's disease (Table 1). Previously, another metal chelator, clioquinol, had been tested to study its effect on the formation of amyloid fibers (Table 1). This drug had been banned in the 1970s because of its suspected association with myelo-optic neuropathy (C. W. Ritchie et al., Arch. Neurol., 2003) and was re-evaluated in this study. In this study, the safety of the product was considered satisfactory for future clinical trials although some adverse effects were reported, and clinical benefits were observed in patients most affected by the disease. Following this trial, a clioquinol derivative (PBT2) was developed and entered into a phase IIa trial (L. Lannfelt et al., Lancet Neurol., 2008). Good treatment tolerance was observed. A decrease in the level of Abeta protein was observed in cephalo-spinal fluid but not in plasma. Two executive functions were also improved in patients who received treatment.

| | | | Number | | |
|---|---|---|---|---|---|
| | | | of | Active | |
| Pathology | Trial name | Phase | patients | substance | Main results |
| Parkinson's | DeferipronPD | II | 22 | Deferiprone | Good treatment |
| | NCT01539837 | | | | tolerance. Decrease |
| | (Completed) | | | | in the amount of |
| | | | | | iron in certain |
| | | | | | areas of the brain. |
| | | | | | Tendency to improve |
| | | | | | motor scores. |
| Parkinson's | Fair-Park I | II/III | 40 | Deferiprone | Good treatment |
| | NCT00943748 | | | | tolerance. Decrease |
| | (Completed) | | | | in the amount of |
| | | | | | iron in the |
| | | | | | substantia nigra. |
| | | | | | Tendency to improve |
| | | | | | motor scores. |
| Parkinson's | Fair-Park II | II/III | — | Deferiprone | — |
| | NCT02655315 | | | | |
| | (Ongoing) | | | | |
| Parkinson's | SKY | II | — | Deferiprone | — |
| | NCT02728843 | | | | |
| | (Ongoing) | | | | |
| Alzheimer's | The 3D Study | II | — | Deferiprone | — |
| | NCT03234686 | | | | |
| Alzheimer's | Ref.: C. W. | II | 36 | Clioquinol | Satisfactory |
| | Ritchie et | | | (PBT1) | treatment tolerance. |
| | al., Arch | | | | Treatment benefit |
| | Neurol., | | | | observed only in |
| | 2003. | | | | the most affected |
| | | | | | patients. |
| Alzheimer's | | | 78 | PBT2 | Good treatment |
| | | | | | tolerance. Decrease |
| | | | | | in the level of |
| | | | | | Abeta protein in the |
| | | | | | cerebrospinal fluid. |

Several iron chelators, such as desferrioxamine, clioquinol, MAO, Vk-28, M30 or M30A (N. Wang et al., Biomacromolecules, 2017), have thus attracted the attention of researchers during preclinical or even clinical trials for the chelation treatment of neurodegenerative diseases. Nevertheless, the efficacy of these molecules and other iron chelators is still limited by their short life-span in the body, their possible cytotoxicity at high doses, their difficulty in crossing the blood-brain barrier and then targeting the most affected area of the brain, and their p prior saturation by endogenous cations.

In parallel with these studies, a clinical trial was requested by the National Institutes of Health (NIH) in 2001 to verify the interest of EDTA for the treatment of cardiovascular diseases using a rigorous scientific protocol. Indeed, it had been postulated in the 1950s that EDTA could chelate the calcium of atherosclerotic plaques, thus causing their degradation. Due to the lack of clinical results, most cardiologists refused this practice. Nevertheless, practitioners continued to use it and in 2007, a study showed that more than 110,000 patients per year in the USA were undergoing this treatment. In 2002, the NIH funded the Trial to Assess Chelation Therapy (TACT, NCT, 00044213), which enrolled 1708 patients aged 50 or older who had suffered a myocardial infarction at least 6 months earlier. The trial showed good tolerability of EDTA therapy in enrolled patients (D. B. Mark et al., Circ. Cardiovasc.

Qual. Outcomes, 2014). A modest but significant effect was observed for patients who underwent EDTA treatment (P. Ouyang et al., Curr. Cardiol. Rep., 2015). However, this effect was much larger for a subgroup of patients with diabetes (633 patients) (E. Escolar et al., Circ.

Cardiovasc. Qual. Outcomes, 2014). Diabetic patients treated with EDTA thus showed a relative risk reduction of 41% for the combined cardiovascular assessment criterium (p<0.001), a 40% (p=0.017) reduction in the risk of non-fatal stroke or non-fatal myocardial infarction and a 43% (p=0.011) reduction in the risk of death. This study thus shows the potential of targeted chelation therapy for a particular group of patients-patients suffering from diabetes—in order to avoid a future stroke.

Currently, it is also being argued that more and more pathologies are linked to dysregulation of metal homeostasis in the body, as has been shown recently for symptoms of cocaine addiction (K. D. Ersche et al., Transl. Psychiatry, 2017) or suspected for syndromes such as autism (D. A. Rossignol et al., Transl Psychiatry, 2014). Many publications have focused on the role of iron, which is visible on MRI, but other endogenous metals such as:
(i) manganese in so-called manganism neurological syndromes (P. Chen et al., J. Neurochem., 2015),
(ii) copper in the case of Wilson's disease; or exogenous metals such as:
(i) mercury for neurotoxic and cardiac damage (J. Ohlander et al., Int. J. Occup. Environ. Health, 2016),
(ii) cadmium, for example in the case of intoxication (V. M. Andrade, Adv. Neurobiol, 2017),
(iii) lead associated with lead poisoning (G.

Bjorklund et al., Arch. Toxicol., 2017), have also been shown to cause severe neurological disorders when their homeostasis is deregulated either by an external factor or a genetic abnormality.

Today, therefore, there is a need to develop novel means of extracting metals from the body, with the aim of preventing and/or treating pathologies related to dysregulation of metal homeostasis, and which would present one or more of the following advantages:
targeted extraction of metals from the body, whether they are present in high or low amounts,
regulation of the homeostasis of essential metals,
an absence of cytotoxicity,
an absence of limited life-span in the body,
facilitated action across the blood-brain barrier in the treatment of neurological diseases,
an application adapted to the prevention and/or treatment of any pathology related to dysregulation of metal homeostasis.

These and other advantages are described in the present disclosure.

### DETAILED DESCRIPTION

In this context, the inventors of the present invention have thus developed a medical device comprising at least one chelator for extracting metal cations.

In a first aspect, the invention thus relates to a device for maintaining metal homeostasis for therapeutic purposes, characterized in that it comprises a means for extracting metal cations.

“Maintaining metal homeostasis for therapeutic purposes” means regulating the level of certain metals within the body, in particular for the purpose of extracting excess metal cations that may be responsible for pathologies. In an embodiment, the term “metal homeostasis” means the homeostasis of metal cations (more specifically the homeostasis of specific metal cations).

In an embodiment, said means for extracting metal cations is selected from:
an implant to which at least one chelator is grafted, or
a perfusion fluid containing at least one chelator. According to the invention, the term “chelator” means an organic group capable of complexing at least one metal cation. According to a preferred embodiment, the chelator is capable of complexing the metal cations that it is desired to extract, and the complexing constant log (K_{C1}) of said chelator for at least one of said metal cations is greater than 10, in particular 11, 12, 13, 14, 15, and is preferably greater than or equal to 15. Advantageously the chelator complexes at least one of the cations of the metals Copper (Cu), Iron (Fe), Zinc (Zn), Mercury (Hg), Cadmium (Cd), Lead (Pb), Aluminum (Al), Manganese (Mn), Arsenic (As), Mercury (Hg), Cobalt (Co), Nickel (Ni), Vanadium (V), Tungsten (W), Zirconium (Zr), Titanium (Ti), Chromium (Cr), Silver (Ag), Bismuth (Bi), Tin (Sn), Selenium (Se), Thallium (Th), Calcium (Ca), Magnesium (Mg), Scandium (Sc), Yttrium (Y), Lanthan (La), Cerium (Ce), Praseodymium (Pr), Neodymium (Nd), Promethium (Pm), Samarium (Sm), Europium (Eu), Gadolinium (Gd), Terbium (Tb), Dysprosium (Dy), Holmium (Ho), Erbium (Er), Thulium (Tm), Ytterbium (Yb), Lutecium (Lu), Actinium (Ac), Thorium (Th), Protactinium (Pa), Uranium (U), Neptunium (Np), Plutonium (Pu), Americium (Am), Curium (Cm), Berkelium, (Bk) Californium (Cf), Einsteinium(Es), Fermium (Fm), Mendelevium (Md), Nobelium (No), and Lawrencium (Lr). Even more advantageously, the chelator complexes at least one of the cations of the metals Copper, Iron, Zinc, Mercury, Cadmium, Lead, Aluminum, Manganese, Magnesium, Calcium, and Gadolinium, especially Manganese and Gadolinium. Even more advantageously, the chelator complexes at least one of the cations of the metals Copper, Iron and/or Zinc. According to the invention, the term “at least one chelator” means the presence of a single type of chelator, a mixture of different chelators or a mixture of several identical chelators.

Advantageously, the specificity of the chelator for said metals (metal cations) to be extracted is high compared with the other cationic trace elements, in particular the difference between the complexing constants is preferably greater than 3, and more particularly the difference between the complexing constants with calcium and magnesium is preferably greater than 3, and even greater than 5.

According to a preferred embodiment, said device also contains trace elements, selected from Calcium, Magnesium, Iron, Copper, Zinc and Manganese, either directly within said polymer, implant or solid, or within the perfusion fluid. This makes it possible, for example, to regulate the homeostasis of essential metals. According to a preferred embodiment, said means of said device makes it possible to extract metal cations from a biological fluid, an organ or a tissue, in particular when the content of said metal cations is less than 1 ppm, in particular 0.1 ppm, 0.01 ppm and is preferably less than 1 ppb. Advantageously, at least more than half of the cations present can be extracted.

According to the invention, the term “biological fluid” means any fluid with which the device of the invention may be brought into contact, such as blood, cerebrospinal fluid, synovial fluid, or peritoneal fluid.

According to the invention, the term “organ” means any organ with which the device of the invention can be brought into contact or into which the device of the invention can be implanted or inserted, such as the brain, liver, pancreas, intestines or lungs.

According to the invention, the term “tissue” means any tissue with which the device of the invention may be brought into contact or into which the device of the invention may be implanted or inserted, such as peritoneum or tumor tissue (where applicable tumor tissue). For example, said device may be brought into contact, inserted or implanted by endoscopy, in particular within a tumor.

According to a preferred embodiment, said means for extracting metal cations, is for example a material, and makes it possible to extract an amount of metal cations representing at least 1% of its mass, and preferably more than 10% of its mass.

### The Means for Metal Extraction is a Dialysis System

Advantageously, and according to a preferred embodiment, the means for extracting metal cations is a dialysis system comprising:
a. a porous dialysis membrane, and
b. a reservoir containing perfusion fluid.

According to the invention, the term “dialysis system” means any system which passes metal cations through an artificial membrane.

According to this specific embodiment, said device is advantageously a microdialysis device. For several years, new technologies for local analyte or sample collection or local drug delivery (microdialysis) have been developed. Microdialysis was developed at the end of the 1950s to recover and deliver different substances in an area of interest (C. M. Kho, Mol. Neurobiol., 2016). Microdialysis makes it possible to collect or deliver only those samples capable of passing through a semi-permeable membrane whose cut-off threshold is chosen according to the intended application. In the case of dialysis, this is often a dynamic diffusion phenomenon, guided by the difference in concentration of the diffusing species between each side of the membrane. In the case of low concentration species, the driving force is often quickly limited or saturated, and the trapping of the species concerned is limited by the equilibrium concentration. Advantageously, the microdialysis device according to the present invention makes it possible to bypass the problems of conventional chelators and to locally extract a very high proportion of the target metal ions, thanks to the maintenance inside a dialysis membrane of the complexing chemical species of at least one target metal. The complexing species are present within macromolecules or nanoparticles that have a mass greater than the membrane cut-off so that the complexing species remain within the liquid (i.e. the perfusion fluid) within the dialysis membrane. The dialysis device containing the complexing species is then placed in the area of interest, for example in the brain for the treatment of neurodegenerative diseases. The cations being smaller than the membrane cut-off will be able to diffuse through the membrane to the solution containing the chelators. The strong complexing properties of the ligands used will allow chelation of the target metals even if they are present in very small amounts. This chelation will therefore reduce the concentration of the free target ions in the solution inside the membrane, thus maintaining a strong concentration gradient of the target metal ion between the concentration on the outside and on the inside of the membrane, prolonging the extraction and maintaining a flow of cations. In order not to disturb the homeostasis of other metal cations, an equivalent concentration of these ions can be placed in the dialysis membrane.

Any microdialysis device known to the person skilled in the art may be used according to the present invention, provided it contains a porous dialysis membrane and a reservoir comprising perfusion fluid containing at least one chelator as mentioned above. In this respect, the cut-off threshold of the porous membrane is lower than the mass of the chelator. By way of example, the devices which may be used in the context of the present invention are the medical devices developed by the company M Dialysis AB, Sweden, such as microdialysis catheters (item numbers 8010509, P000049, 8010337, this list not being exhaustive).

According to this preferred embodiment, the perfusion fluid is a colloidal suspension of nanoparticles whose mean diameter is greater than the pores of said porous dialysis membrane, said nanoparticles comprising as active principle at least one chelator. In one aspect, the cut-off threshold of the porous dialysis membrane is less than the mass of the chelator, i.e. the mass of the nanoparticle comprising at least one chelator. the perfusion fluid is a colloidal Alternatively, suspension of polymers whose mean diameter is greater than the pores of said dialysis membrane, said polymers being grafted to an active principle which is at least one chelator. In this respect, the cut-off threshold of the porous dialysis membrane is lower than the mass of the chelator, i.e. the mass of the polymer to which at least one chelator is grafted.

According to the invention, the term “colloidal suspension” means a mixture of liquid and solid, insoluble particles that remain dispersed evenly, the particles often being sufficiently small (microscopic or nanoscopic) to keep the mixture stable and homogeneous. According to an embodiment, said mean diameter is larger than the pores of said dialysis membrane by at least 10%, 20%, 30%, 40%, 50%, 60%, 70%, 80%, 90% or 100%. According to the invention, the term “mean diameter” means the harmonic mean of the diameters of nanoparticles or polymers to which at least one chelator is grafted. The size distribution of nanoparticles or polymers is, for example, measured using a commercial particle size analyzer, such as a photon correlation spectroscopy (PCS)-based Malvern Zeta Sizer Nano-S particle size analyzer, which is characterized by a mean hydrodynamic diameter. A method for measuring this parameter is also described in ISO 13321:1996.

In an embodiment, the colloidal suspension contains more than 1% by mass of nanoparticles or polymers, in particular more than 28, 3%, 48, 5%, 6%, 7%, 8%, 9%, and preferably more than 10% by mass.

### Nanoparticles that can be Used in a Device, in Particular a Dialysis System or an Implant, According to the Present Invention

The nanoparticles that can be used in the present invention have two essential features:
they are polysiloxane-based or silica-based,
they have a mean diameter greater than 3 nm, preferably less than 50 nm.

In an embodiment said nanoparticle comprises as active principle at least one chelator capable of complexing metal cations, said chelator having a complexing constant log (K_{C1}) for at least one of said metal cations is greater than 10, and preferably greater than or equal to 15. According to the invention, the term “silica-based nanoparticles” means nanoparticles characterized by a silica mass percentage of at least 8%.

According to the invention, the term “polysiloxane-based nanoparticles” means nanoparticles characterized by a silicon mass percentage of at least 8%.

According to the invention, the term “polysiloxane” means an inorganic cross-linked polymer consisting of a chain of siloxanes.

The structural units of the polysiloxane, identical or different, are of the following formula:

Si(OSi)ₙR₄₋ₙ

wherein:
R is an organic molecule linked to the silicon by a covalent Si—C bond
n is an integer between 1 and 4.

As a preferred example, the term “polysiloxane” includes in particular polymers resulting from the condensation by the sol-gel process of tetraethylorthosilicate (TEOS) and aminopropyltriethoxysilane (APTES).

Advantageously, said nanoparticle thus comprises:
a. polysiloxanes, with a silicon mass ratio of at least 8% of the total mass of the nanoparticle, preferably between 8% and 50% of the total mass of the nanoparticle,
b. chelators, preferably in a proportion between 5 and 1000, and preferably between 5 and 100 per nanoparticle,
c. if need be, metallic elements, for example in a proportion between 5 and 100, preferably between 5 and 20 per nanoparticle, said metallic elements being complexed to the chelators.
   Even more advantageously, said nanoparticle has the following formula (I):

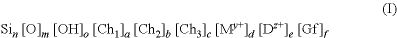

wherein:
n is between 20 and 50,000 preferably between 50 and 1000.
m is greater than n and less than 4 n
is between 0 and 2 n
Ch₁, Ch₂ and Ch₃ are chelators, identical or different, linked to the Si of the polysiloxanes by a covalent Si—C bond; a, b and c are integers between 0 and n and a+b+c is less than or equal to n, preferably a+b+c is between 5 and 100, for example between 5 and 20,
M^{y+} and D^{z+} are metal cations, identical or different, with y and z=1 to 6; d and e are integers between 0 and a+b+c, and d+e is less than or equal to a+b+c,
Gf are targeting grafts, identical or different, each linked to Si by an Si—C bond and derived from the grafting of a targeting molecule allowing the targeting of nanoparticles to biological tissues of interest, for example to tumor tissues, f is an integer between 0 and n.

In an embodiment, the nanoparticles usable according to the present invention do not comprise metallic elements.

In other words, in the above definition, said nanoparticle comprises only the elements a. (polysiloxanes or silica) and b. (chelators).

In an embodiment, the chelators complex the cations of the metals Cu, Fe, Zn, Hg, Cd, Pb, Mn, Al, Ca, Mg, Gd. In an embodiment, the chelators are obtained by grafting (covalent bonding) onto the nanoparticle one of the following complexing molecules or its derivatives, such as polyamino polycarboxylic acids and derivatives thereof, in particular selected from: DOTA (1,4,7,10-tetraazacyclododecane-N, N′,N″, N″-tetraacetic acid), DTPA (diethylene triamine pentaacetic acid), DO3A-pyridine of formula (I) below:

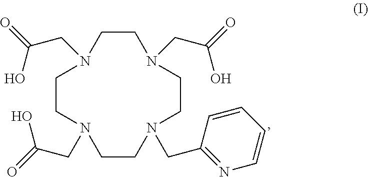 [SMILES: O=C(O)CN1CCN(CC(=O)O)CCN(Cc2ccccn2)CCN(CC(=O)O)CC1]

EDTA (2,2′, 2″, 2″-(ethane-1,2-diyldinitrilo)tetraacetic acid), EGTA (ethylene glycol-bis (2-aminoethylether)-N, N,N′,N′-tetraacetic acid), BAPTA (1,2-bis (o-aminophenoxy) ethane-N, N,N′,N′-tetraacetic acid), NOTA (1,4,7-triazacyclononane-1,4,7-triacetic acid), DOTAGA ((2-(4,7,10-tris(carboxymethyl)-1,4,7,10-tetraazacyclododecan-1-yl) pentanedioic acid), DFO (deferoxamine), amide derivatives such as for example DOTAM (1,4,7,10-tetrakis (carbamoylmethyl)-1,4,7,10 tetraazacyclododecane) or NOTAM (1,4,7-tetrakis (carbamoylmethyl)-1,4,7-triazacyclononane), as well as mixed carboxylic acid/amide derivatives, phosphonic derivatives such as DOTP (1,4,7,10-tetraazacyclododecane-1,4,7,10-tetrakis (methylene phosphonate)) or NOTP (1,4,7-tetrakis (methylene phosphonate)-1,4,7-triazacyclononane), cyclam derivatives such as TETA (1,4,8,11-tetraazacyclotetradecane-N, N′,N″, N″-tetraacetic acid), TETAM (1,4,8,11-tctraazacyclotetradecane-N,N′,N″, N″-tetrakis (carbamoylmethyl)), TETP (1,4,8,11-tetraazacyclotetradecane-N, N′,N″, N″-tetrakis (methylene phosphonate)) or mixtures thereof.

Preferably, the above said chelators are directly or indirectly covalently linked to the silicas of the polysiloxanes of the nanoparticle. The term “indirect” linking refers to the presence of a molecular “linker” or “spacer” between the nanoparticle and the chelator, said linker or spacer being covalently bonded to one of the constituents of the nanoparticle.

According to a preferred embodiment, said nanoparticle is a polysiloxane-based nanoparticle with a mean diameter between 3 and 50 nm, comprising the chelator obtained by grafting DOTA, DOTAGA or DTPA onto the nanoparticle. According to a preferred embodiment, said nanoparticle is a polysiloxane-based nanoparticle with a mean size greater than 20 kDa and less than 1 MDa, comprising the chelator obtained by grafting DOTA, DOTAGA or DTPA onto the nanoparticle.

In a preferred embodiment, said colloidal suspension comprising said nanoparticles also contains trace elements selected from Calcium, Magnesium, Iron, Copper, Zinc, or Manganese.

The nanoparticles according to the present invention can be obtained by the process described in patent application FR1053389.

### Polymers that can be Used in a Device According to the Present Invention

In another embodiment of the invention, polymers may be used instead of the above-mentioned nanoparticles. In such a case, said polymers are grafted to at least one chelator.

According to the invention, the term “polymer” means any macromolecule formed by the covalent sequence of a very large number of repeating units derived from one or more monomers. The polymers preferably used in the present invention are for example of the family of chitosans, polyacrylamides, polyamines or polycarboxylic acids. For example, they may be polymers containing amino functions such as chitosan. According to a preferred embodiment, said polymer is biocompatible.

According to an embodiment, the chelators or derivatives thereof grafted onto said polymers are polyamino polycarboxylic acids and derivatives thereof, in particular selected from: DOTA, DTPA, DO3A-pyridine of formula (I) above, EDTA, EGTA, BAPTA, NOTA, DOTAGA, DFO, DOTAM, NOTAM, DOTP, NOTP, TETA, TETAM and TETP or mixtures thereof.

Preferably, the above said chelators are linked directly or indirectly by covalent bond to the polymer or to a polymer chain of more than 10 kDa and preferably more than 100 kDa. The term “indirect” linking means the presence of a molecular “linker” or “spacer” between the polymer and the chelator, said linker or spacer being covalently bonded to one of the constituents of said polymer.

In an embodiment, the chelators or derivatives thereof grafted onto said polymers will comprise dithiocarbamate functions.

According to a preferred embodiment, said polymer grafted with a chelator is selected from: chitosan grafted with DPTA-BA or chitosan grafted with DFO.

In a preferred embodiment, said colloidal suspension comprising said polymers also contains trace elements, selected from Calcium, Magnesium, Iron, Copper, Zinc, or Manganese.

### Chelating Molecules that can be Used in a Device According to the Present Invention

Alternatively, the perfusion fluid is a solution of chelating molecules. Said chelating molecules may have a mean diameter greater than the pores of said dialysis membrane, i.e. greater than the membrane cut-off threshold in order to be maintained within the dialysis membrane fluid, or they may have a mean diameter less than the pores of said porous dialysis membrane, in which case they may pass through the pores of the membrane before passing into the body and be naturally eliminated by the kidneys or liver.

In this embodiment, said chelating molecules have a complexing constant log (Kc) for at least one of said metal cations greater than 10, and preferably greater than or equal to 15.

In this embodiment, said solution of chelating molecules also contains trace elements, selected from Calcium,

### Magnesium, Iron, Copper, Zinc or Manganese.

### Chelator-Grafted Implants that can be Used in a Device According to the Present Invention

Alternatively, the means for extracting metal cations is an implant comprising at least one chelator. According to an embodiment, the means for extracting metal cations is an implant on which at least one chelator is grafted. According to the invention, an “implant” means any element intended to be introduced into the body. It may be “polymers” or “any other solid” as described in the present description.

In this embodiment, polymers such as those mentioned above can be used in a perfusion fluid.

According to the invention, the term “any other solid” means, without being restrictive, ceramic, metallic, composite, solid or porous parts, optionally surface functionalized or not surface functionalized, and which may have different shapes (such as balls, tubes, plates, etc.).

In this embodiment, said implant can be implanted, in particular temporarily, and then extracted. Preferentially, said implant can be implanted within the brain, liver, pancreas, etc., of the subject to be prevented and/or treated. Said implant can be resorbable and naturally be progressively eliminated by the body. Said implant may also include at least one chelator which diffuses slowly in the body, for example a diffusion of less than 100 mg of chelating molecules released per day, and preferably less than 10 mg/day and/or allowing a diffusion of less than 1% of the total mass per day. Said implant may be placed in direct contact with the tissues or under the skin. Alternatively, said implant may be in a reservoir with a dialysis fluid in contact with the subject to be treated.

In a second aspect, the present invention relates to the use of a colloidal suspension as mentioned above, in particular for use in a device such as those mentioned above.

The invention thus relates to a colloidal suspension of nanoparticles comprising an active principle, for use for therapeutic purposes, characterized in that it is contained in a device for maintaining metal homeostasis comprising a porous dialysis membrane, and in that the mean diameter of said nanoparticles is greater than the pores of the porous dialysis membrane of said device. Advantageously, said device is a microdialysis device.

In an embodiment, the invention also relates to a colloidal suspension of polymers grafted to an active for principle, for use therapeutic purposes, characterized in that it is contained in a device for maintaining metal homeostasis comprising a porous dialysis membrane, and in that the mean diameter is greater than the pores of said porous dialysis membrane, said polymers being grafted to an active principle. Advantageously, said device is a microdialysis device. In an embodiment, the invention relates to a device for maintaining metal homeostasis as claimed in any one of claims 1 to 13, characterized in that said device comprises means allowing it to be placed in contact, through a dialysis membrane, or implanted within:
a biological fluid, such as blood, cerebrospinal fluid, synovial fluid or peritoneal fluid, or
an organ, such as the brain, liver, pancreas, intestines or lungs, or
a tissue, such as the peritoneum or tumor tissue.

According to a preferred embodiment, the invention relates to a colloidal suspension mentioned above for use in maintaining metal homeostasis.

According to another preferred embodiment, the invention relates to a colloidal suspension mentioned above for use in the treatment of neurological disease or cerebral degeneration, such as Parkinson's disease, Alzheimer's disease, neurodegeneration with brain iron accumulation (NBIA, also called neurodegeneration with brain iron overload), Wilson's disease, or Huntington's disease. According to another preferred embodiment, the invention relates to a colloidal suspension mentioned above for use in the treatment of autism.

According to another preferred embodiment, the invention relates to a colloidal suspension mentioned above for use in the treatment of type II diabetes or cardiovascular disease.

According to another preferred embodiment, the invention relates to a colloidal suspension mentioned above for use in the treatment of tumors.

In a third aspect, the present invention relates to the use of a nanoparticle as mentioned above, in particular for use in a device such as those mentioned above.

In an embodiment, the invention thus relates to a polysiloxane-based nanoparticle having a diameter greater than 3 nm, preferably less than 50 nm, for use for therapeutic purposes in a device for maintaining metal homeostasis, said nanoparticle comprising as active principle at least one chelator capable of complexing the metal cations, and characterized in that its complexing constant log (K_{C1}) for at least one of said metal cations is greater than 10, and preferably greater than or equal to 15. Advantageously, said device is a microdialysis device.

According to a preferred embodiment, the invention relates to a nanoparticle mentioned above for use in maintaining metal homeostasis.

In another preferred embodiment, the invention relates to the above-mentioned nanoparticle for use in the treatment of neurological diseases or brain degeneration, such as NBIA type diseases, Parkinson's disease, Alzheimer's disease, Wilson's disease or Huntington's disease.

In another preferred embodiment, the invention relates to a nanoparticle mentioned above for use in the treatment of autism.

In another preferred embodiment, the invention relates to the above-mentioned nanoparticle for use in the treatment of type II diabetes or cardiovascular diseases. In another preferred embodiment, the invention relates to the above-mentioned nanoparticle for use in the treatment of tumors.

In a fourth aspect, the present invention relates to the use of a polymer as mentioned above, in particular for use in a device such as those mentioned above.

In an embodiment, the invention thus relates to a polymer, for use for therapeutic purposes in a device for maintaining metal homeostasis, said polymer being grafted to at least one chelator capable of complexing the metal cations, and characterized in that its complexing constant log (K_{C1}) for at least one of said metal cations is greater than 10, and preferably greater than or equal to 15. Advantageously, said device is a microdialysis device.

According to a preferred embodiment, the invention relates to a polymer mentioned above for use in maintaining metal and/or protein homeostasis.

According to another preferred embodiment, the invention relates to a polymer mentioned above for use in the treatment of neurological diseases or cerebral degeneration, NBIA type diseases, Parkinson's disease, Alzheimer's disease, Wilson's disease or Huntington's disease.

According to another preferred embodiment, the invention relates to a polymer mentioned above for use in the treatment of autism.

According to another preferred embodiment, the invention relates to a polymer mentioned above for use in the treatment of type II diabetes or cardiovascular diseases. According to another preferred embodiment, the invention relates to the above-mentioned polymer for use in the treatment of tumors.

The present invention also relates to a method for extracting metal cations from a subject comprising the administration of an implant having at least one chelator grafted thereon, or the use of a perfusion fluid containing at least one chelator within a device such as those mentioned above.

According to the invention, said “subject” means a human or animal to which prevention or treatment is provided. The invention will be best illustrated by the following examples and figures. The following examples are intended to clarify the subject matter of the invention and to illustrate advantageous embodiments, but are in no way intended to restrict the scope of the invention.

### FIGURES

FIG. 1 shows the image obtained at the end of the perfusion of the MnCl: solution. This is a coronal section at the microdialysis membrane (black dot). The highlight around the membrane corresponds to the presence of Mn²⁺ (positive MRI contrast agent).

FIG. 2 shows the image obtained at the end of the perfusion with the nanoparticle suspension. This is a coronal section at the microdialysis membrane (black dot). The highlight around the membrane corresponds to the presence of Mn²⁺ (positive MRI contrast agent).

FIG. 3 shows the image corresponding to the difference between the two previous images (shown in FIGS. 1 & 2) and highlighting the decrease in tissue concentration in Mn²⁺ (highlighted at the microdialysis probe).

FIG. 4 shows the image obtained at the end of the perfusion with the MnCl: solution. This is a coronal section at the microdialysis membrane (black dot). The highlight around the membrane corresponds to the presence of Mn²⁺ (positive MRI contrast agent).

FIG. 5 shows the image obtained at the end of the perfusion with saline. This is a coronal section at the microdialysis membrane (black dot). The highlight around the membrane corresponds to the presence of Mn²⁺ (positive MRI contrast agent).

FIG. 6 shows the image corresponding to the difference between the two previous images (shown in FIGS. 4 & 5) and highlighting the absence of decrease in tissue concentration in Mn²⁺ (almost no highlighting at the microdialysis probe).

FIG. 7 shows the MRI image of solutions 1, 2, 3, 4 and 5.

FIG. 8: Hydrodynamic diameter of the nanoparticles obtained in Example 7.

FIG. 9: Hydrodynamic diameter of the nanoparticles obtained in Example 8.

### EXAMPLES

### Example 1: Extraction of manganese ions from rodent brain

The study was conducted on male Wistar rats (weight: 250 g).

On Day 0, for the insertion of the microdialysis cannula, the animal is placed under gas anesthesia (2.5% isoflurane under O₂/N₂ (80:20)) with the use of a heating mat used during the procedure and the recovery phase. Prior to incision of the skin to clear the skull, local anesthesia with subcutaneous injection of lidocaine (Xylovet 21.33 mg/mL) is performed (4 mg/kg diluted in 0.9% NaCl with an injected volume of 10 μL/g). After incision of the skin, the skull is cleared in order to position a micro drill (diameter <1 mm) r skull piercing. The insertion of the probe is done under stereotaxy. The dialysis cannula (diameter <500 μm) is gently inserted into the brain at the desired position and depth. After positioning the cannula, a fast-setting fixing resin is applied and screwed onto the animal's skull. The skin is then sewn back together to close the wound. Before the animal wakes up, an analgesic (Buprecare) is administered subcutaneously.

Administration of the analgesic is repeated at intervals of 8 to 12 hours for 2 days following the insertion of the microdialysis probe. In order to limit dehydration of the animal, a subcutaneous injection of 0.9% NaCl (about 0.5 mL for mice, 5 mL for rats) is carried out at the beginning of the procedure. To prevent dry eye, an ophthalmological ointment (Liposic) is applied at the beginning of the procedure.

The MRI spectroscopy and imaging protocol is performed on Day 3. The protocol is performed on animals under gas anesthesia (2.5% isoflurane under O₂/N₂=(80:20)) with the use of a heating mat used during the procedure and the recovery phase and with breath control during NMR acquisitions. Before positioning the animal in the MRI (Bruker Biospin 4.7 Tesla), the microdialysis probe (2 mm long membrane, 6 kDa cut-off, CMA Microdialysis AB, is inserted into the microdialysis Kista, Sweden) cannula. An MRI surface antenna (Doty Scientific, 8 mm diameter, used for transmission and reception, is positioned on the skull of the animal vertically to the microdialysis probe. The MRI acquisitions (T1-weighted Flash sequence, echo time 2 ms, repetition time 150 ms, coronal sections, section thickness 1 mm, acquisition time 3 minutes) are performed continuously during the perfusion of the microdialysis probe.

### Results

### Example 1A

The microdialysis probe is perfused with a 1 mM MnCl: solution in saline at a flow rate of 10 μL/min for 30 minutes. The microdialysis probe is then perfused with a suspension of polysiloxane nanoparticles with free DOTAGA on their surface (28.1 mg diluted in 1 mL saline+100 μL of NaOH and HCl to equilibrium at pH 7= or 28.1 mg in total volume of 1100 μL) at a flow rate of 10 μL/min for 30 minutes. The polysiloxane nanoparticles used consist of a polysiloxane matrix to which are grafted cyclic chelators of DOTAGA. These nanoparticles have a hydrodynamic diameter of 11.5±6.3 nm. This size prevents their passage through the dialysis membrane, whose pore diameter is 2 to 3 nm.

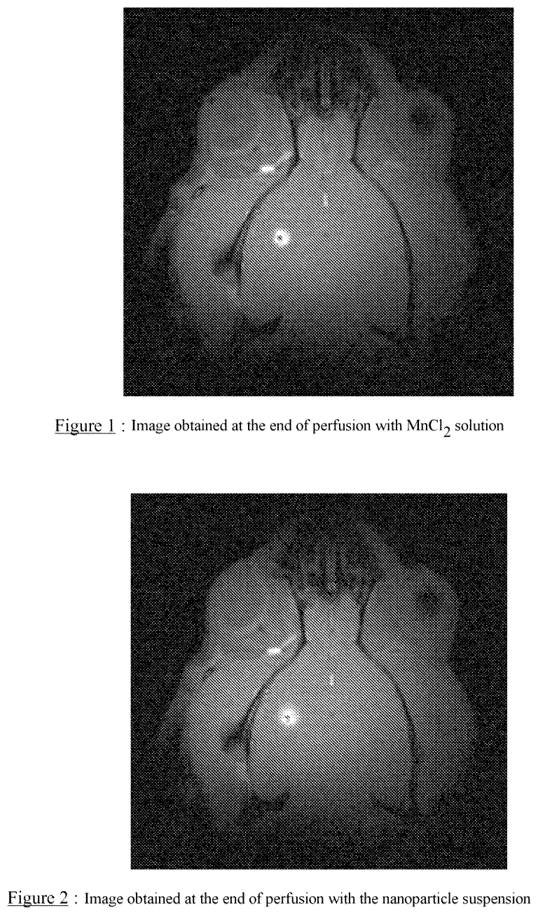

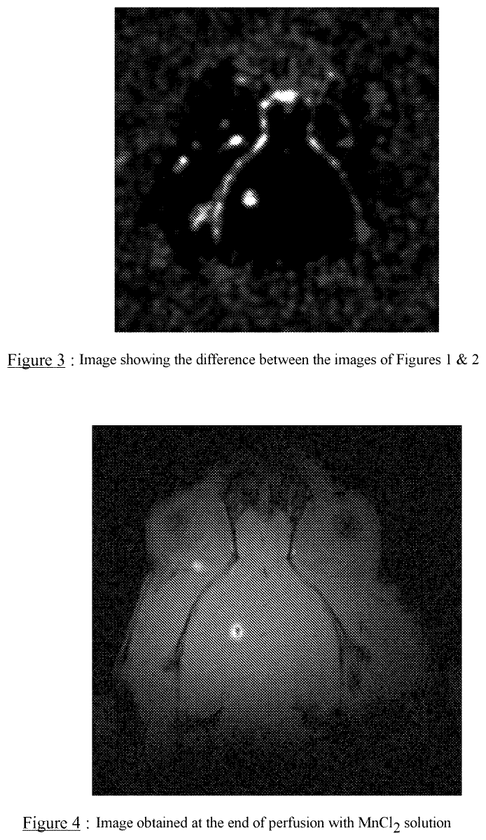

The image obtained at the end of the perfusion of the MnCl₂ solution is shown in FIG. 1, and the image obtained at the end of the perfusion with the nanoparticle suspension is shown in FIG. 2. FIG. 3 shows the image corresponding to the difference between the two previous images and highlighting the decrease in tissue Mn²⁺ concentration (highlighted at the microdialysis probe).

### Example 1B

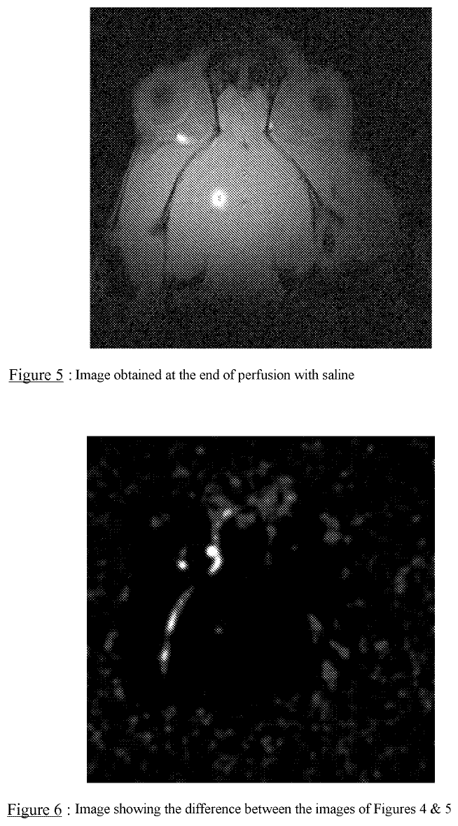

The microdialysis probe is perfused with a 1 mM MnCl₂ solution in saline at a flow rate of 10 μL/min for 30 minutes. The microdialysis probe is then perfused with saline at 10 μL/min for 30 minutes. The image obtained at the end of perfusion of the MnCl: solution is shown in FIG. 4, and the image obtained at the end of perfusion with saline is shown in FIG. 5. FIG. 6 shows the image corresponding to the difference between the two previous images and showing the absence of a decrease in tissue Mn²⁺ concentration (almost no highlighting at the microdialysis probe).

### CONCLUSIONS

MRI allows the objectification of tissue concentration variations in Mn²⁺ cation (paramagnetic MRI contrast agent). The presence of chelating nanoparticles in the perfusate results in a significant decrease in intensity in MRI sections due to a decrease in local tissue Mn²⁺ concentration. This decrease in intensity is not observed in the absence of chelating nanoparticles.

### Example 2: Extraction of Intra-Tissue Gadolinium by Perfusion of Nanoparticle Solution

The study was conducted on male Wistar rats (weight: 250 g).

On Day 0, for the insertion of the microdialysis cannula, the animal is placed under gas anesthesia (2.5% isoflurane under O₂/N₂=(80:20)) with the use of a heating mat used during the procedure and the recovery phase. Prior to incision of the skin to clear the skull, local anesthesia with subcutaneous injection of lidocaine (Xylovet 21.33 mg/mL) is performed (4 mg/kg diluted in 0.9% NaCl with an injected volume of 10 μL/g). After incision of the skin, the skull is cleared in order to position a micro drill (diameter <1 mm) for the drilling of the skull. The insertion of the probe is done under stereotaxy. The dialysis cannula (diameter <500 μm) is gently inserted into the brain at the desired position and depth. After positioning the cannula, a fast-setting fixing resin is applied and screwed onto the animal's skull. The skin is then sewn back together to close the wound. Before the animal wakes up, an analgesic (Buprecare) is administered subcutaneously. Administration of the analgesic is repeated at intervals of 8 to 12 hours for 2 days following the insertion of the microdialysis probe. In order to limit dehydration of the animal, a subcutaneous injection of 0.9% NaCl (about 0.5 mL for mice, 5 mL for rats) is carried out at the beginning of the procedure. To prevent dry eye, an ophthalmological ointment (Liposic) is applied at the beginning of the procedure.

The microdialysis perfusion protocol is performed on Day 3. The protocol is performed on animals under gas anesthesia (2.5% isoflurane under O_/N: (80:20)) with the use of a heating mat used during the procedure and the recovery phase and with control of the respiratory frequency. The microdialysis probe (2 mm long membrane, 6 kDa cut-off, CMA Microdialysis AB, Kista, Sweden) is inserted into the microdialysis cannula and the perfusion is performed at a flow rate of 10 μL/min.

Perfusion is carried out over 30 minutes with a perfusate consisting of saline supplemented with 1 mM GdC13 (solution 1). The dialysate is collected at the end of microdialysis (solution 2).

The microdialysis probe is then perfused with a nanoparticle suspension VL29-5 (28.1 mg diluted in 1 mL saline+100 μL NaOH and HCl to equilibrate at pH 7= or 28.1 mg in a total volume of 1100 μL) for 30 minutes (solution 3). The dialysate is collected at the end of microdialysis (solution 4). The nanoparticles used are identical to those in Example 1, i.e. they have a hydrodynamic diameter of 11.5+6.3 mm. This size prevents their passage through the dialysis membrane, which has a pore diameter of 2 to 3 nm.

These 4 solutions (as well as a saline solution 5) are imaged in a 4.7 Tesla MRI with a T1-weighted gradient echo sequence (repetition time 40 ms, echo time 2.6 ms, tilt angle)80°.

The images of the 5 tubes are shown in FIG. 7.

### Results

The results in FIG. 7 thus highlight the increase in intensity of solution 4 compared to solution 5, which clearly shows the uptake and chelation of tissue Gd during the passage of the nanoparticle solution through the microdialysis probe.

### Example 3: Synthesis of chitosane-DTPA-BA

The chitosan used has a mean molecular weight of 200 kDa. DTPA-BA (diethylenetriaminepentaacetic dianhydride) was supplied by Chematech, Dijon, France and used as such. The VIVAFLOW cassettes were purchased from Sartorius and used as is. The perfusion fluid was purchased from Phymep (Perfusion Fluid CNS Sterile, item number P000151) and used as is.

A mass of 0.5 g of chitosan was weighed and inserted into a 500 mL container. A volume of 250 mL of distilled water was added and the solution was stirred. Using a pH meter and a 50% acetic acid solution, the pH was adjusted to 4.0+0.1. The solution was stirred for 24 h. At 24 h the pH was again adjusted to 4.0±0.1. This procedure was repeated until all the chitosan was completely dissolved.

A mass of 5.36 g of DTPA-BA was weighed and added to the resulting solution. The solution was stirred for 48 h. At 48 h the solution was purified using a Vivaflow cassette with a 100 kDa cut-off until a purification rate of at least 100,000 was achieved. Again using a Vivaflow cassette, the solvent is replaced by the CNS perfusion fluid at the same concentration.

### Example 4: Synthesis of Chitosan-DFO

The chitosan used has a mean molecular weight of 200 kDa. p-NCS-Bz-DFO (N1-hydroxy-N1-(5-(4-(hydroxy (5-(3-(4-isothiocyanatophenyl) thioureido) pentyl) amino)-4-oxobutanamido) pentyl)-N4-(5-(N-hydroxyacetamido) pentyl) succinamide) was purchased from Chematech Mdt and used as is. VIVAFLOW cassettes were purchased from Sartorius and used as is. The perfusion fluid was purchased from Phymep (Perfusion Fluid CNS Sterile, item number P000151) and used as is.

A mass of 0.5 g of chitosan was weighed and placed in a 500 mL container. A volume of 250 mL of distilled water was added and the solution was stirred. Using a pH meter and a 50% acetic acid solution, the pH was adjusted to 4.0+0.1. The solution was stirred for 24 h. At 24 h the pH was again adjusted to 4.0+0.1. This procedure was repeated until all the chitosan was completely dissolved. A 500 mg mass of p-NCS-Bz-DFO was weighed and added to the resulting solution. The solution was stirred for 48 h. At 48 h the solution was purified using a Vivaflow cassette with a 100 kDa cut-off until a purification level of at least 100,000 was reached. Again using a Vivaflow cassette, the solvent is replaced by the CNS perfusion fluid at the same concentration.

### Example 5: MetalSorb Purification and Conditioning

The polyacrylamide polymer containing dithiocarbamate functions, Metalsorb FZ, was supplied by SNF, France and used as is. VIVAFLOW cassettes were purchased from Sartorius and used as is. The perfusion fluid was purchased from Phymep (Perfusion Fluid CNS Sterile, item number P000151) and used as is.

A volume of 50 ml of Metalsorb 20% w/w was measured and placed in a 250 mL container. A volume of 150 ml of water was added and the solution was stirred for two hours. At 2 h, the solution was purified using a Vivaflow cassette with a 100 kDa cut-off until a purification rate of at least 100,000 was achieved. Again using a Vivaflow cassette, the solvent was replaced by CNS perfusion fluid at equal concentration.

### Example 6: Use of Materials Obtained in Examples 3, 4 and 5

The materials obtained in Examples 3, 4 and 5 above may be advantageously used as a means for extracting metal cations according to the present invention. The solutions can be used directly or by adapting the formulation to form a perfusion fluid, or the polymers can be extracted and consolidated to form a macroscopic solid which can be implanted.

### Example 7: Synthesis of Polysiloxane-EDTA Nanoparticles

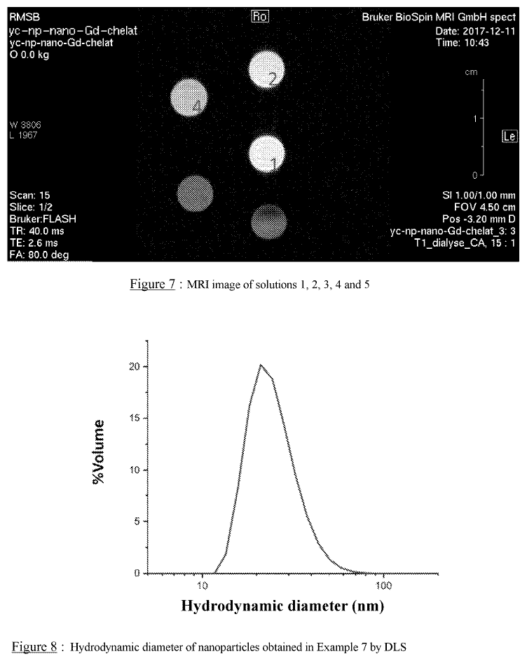

Polysiloxane particles comprising EDTA (ethylenediaminetetraacetate) type chelates Si@EDTA are obtained by mixing three silane precursors: (i) TEOS (tetraethylorthosilicate-((Si(OC₂H₅)₄, 98%-Sigma-Aldrich Chemicals, France)), (ii) APTES (3 (aminopropyl)triethoxy silane-(H₂N(CH₂)₃—Si(OC₂H₅)₃, 99%-Sigma-Aldrich Chemicals, France)) and (iii) Si-EDTA (N-(trimethoxysilylpropyl)ethylenediaminetriacetic acid, trisodium salt (N-[3-trimethoxysilylpropyl]ethylenediamine triacetic acid trisodium salt at 45% in water, ABCR, Germany)). The 3 precursors are placed in DEG (diethylene glycol-DEG, 99%-SDS Carlo Erba (France)) with a molar ratio 2:1:3 (TEOS/APTES/Si-EDTA). The mixture is kept under stirring at room temperature for 30 minutes before adding a 3 times higher volume of water and a new stirring phase of 17 hours at the same temperature. The temperature is then raised to 80° C. and stirring is maintained for 6 hours (the pH is adjusted to a value of 7.4 after two hours of heating). The heating is then switched off and the solution is kept under stirring for 17 hours. The solution is then purified by tangential filtration. The nanoparticles have a hydrodynamic diameter of 21+9 nm in dynamic light scattering (DLS) using a PCS-based Malvern Zeta Sizer Nano-S particle size analyzer (FIG. 8).

### Example 8: Synthesis of Polysiloxane-DTPA Nanoparticles

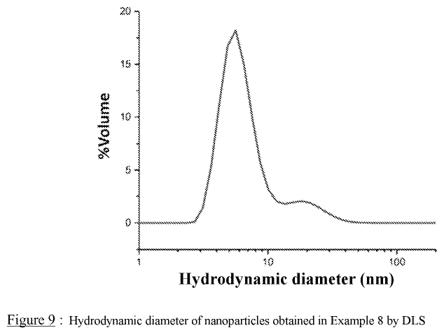

For nanoparticles comprising chelates of the DTPA (diethylenetriaminepentaacetic acid) type, a preliminary step is necessary to graft the chelate onto a silane. The silane comprising DTPA is obtained by reacting a derivative of DTPA: DTPA-BA (diethylenetriaminepentaaceticacid dianhydride-CheMatech, Dijon, France) with APTES in DEG in a ratio of 1:1 DTPA-BA/APTES. The solution is left under stirring for 24 hours. Then TEOS is added with a 3:1:1 TEOS/APTES/DTPA-BA ratio. After 1 hour under stirring in DEG, water is added (10 times the volume of DEG used). The solution is then stirred for 24 hours at room temperature, heated to 50° C. and stirred again for 24 hours. Finally, the solution is cooled to room temperature and left to stir for 72 hours. The nanoparticles are then purified by tangential filtration and the pH is raised to 7.4. The nanoparticles have a hydrodynamic diameter of 7+3 nm in DLS, evaluated using a PCS-based Malvern Zeta Sizer Nano-S particle size analyzer, with a second population at 20+7 nm (FIG. 9).

### Example 9: Comparison of Microdialysis Flow Rates on Metal Extraction

The extractability of a perfusion fluid comprising a chelator in a microdialysis device from an aqueous solution comprising several metal cations was evaluated in this example.

Several flow rates (1, 2 and 5 μL/min) were tested using the same perfusion fluid (polysiloxane-EDTA nanoparticles whose synthesis is described in Example 8 at an EDTA concentration of 15 mM dispersed in water). The microdialysis membrane (63 Microdialysis Catheter, M Dialysis AB, Sweden) had a cut-off of 20 kDa. The solution used to test the chelating ability of the perfusion fluid was an aqueous solution comprising Al (III), Cd (II), Zn (II), Cu (II) and Pb (II) ions each at a concentration of 100 ppb. The pH of the solution was adjusted to 7.4 and HEPES (4-(2-hydroxyethyl)-1-piperazineethanesulfonic acid, Sigma-Aldrich Chemicals (France) was added as a buffer at a concentration of 1.2 g/L. The total volume of the solution is 600 mL. Microdialysis extraction took 40 min at flow rates of 2 and 5 μL/min. The sample at 1 μL/min was obtained in 100 minutes. These samples were analyzed by ICP/MS and the amounts of each metal were reported in Table 2. This experiment was replicated 4 times and shows a better extraction for each of the metals using the perfusion fluid based on chelating nanoparticles compared to a conventional microdialysis use where the perfusion fluid initially contains only water (H₂O) under all conditions tested. Metal uptake at concentrations above their “diffusion concentration” in the medium to be purified was observed in the case of the use of a perfusion fluid comprising a chelator. Chelation is particularly effective for aluminum because of its smaller size, which allows faster diffusion through the membrane. A flow rate of 2 μL/min appears to be a good compromise between efficient extraction and sample amount and has been selected for Examples 10 and 11.

TABLE 2
Concentration of extracted metals in the perfusion
fluid by comparing water and polysiloxane-EDTA
nanoparticles (15 mM) at different flow rates.

| Perfusion | Flow rate | Al | Cu | Cd | Pb |
|---|---|---|---|---|---|
| Fluid | (μL · min⁻¹) | (ppb) | (ppb) | (ppb) | (ppb) |
| H₂O | 5 | 15 | 32 | 24 | 33 |
| H₂O | 2 | 32 | 55 | 29 | 83 |
| H₂O | 1 | 72 | 55 | 61 | 86 |
| Polysiloxane- | 5 | 55 | 51 | 50 | 51 |
| EDTA | | | | | |
| Polysiloxane- | 2 | 436 | 85 | 105 | 95 |
| EDTA | | | | | |
| Polysiloxane- | 1 | 292 | 106 | 67 | 109 |
| EDTA | | | | | |

### Example 10: Comparison of Perfusion Fluids Based on Polysiloxane-DTPA and Polysiloxane-EDTA Nanoparticles

The relative efficiency of the nanoparticles obtained in Examples 7 and 8 was compared using the same metal mixture as described in Example 9 with a microdialysis flow rate of 2 μL/min⁻¹ and a sample collection time of 40 min with a microdialysis membrane having a cut-off of 20 kDa. Table 3 summarizes the results obtained using 3 different perfusion fluids: (i) water, (ii) polysiloxane-EDTA nanoparticles and (iii) polysiloxane-DTPA nanoparticles at a chelator concentration of 15 mM. DTPA-based nanoparticles have a very high aluminum extraction capacity due to the very high affinity of the chelator for this species. The presence of aluminum seems to saturate the surface chelators reducing the efficiency of the fluid for other metals. The polysiloxane-DTPA nanoparticles make it possible to obtain a very specific perfusion fluid for the extraction of aluminum.

TABLE 3
Concentration of extracted metals in the perfusion fluid
comparing water and polysiloxane-EDTA and polysiloxane-
DTPA nanoparticles (15 mM) at a flow rate of 2 μL/min⁻¹.

| Perfusion | Flow rate | Al | Cu | Cd | Pb |
|---|---|---|---|---|---|
| Fluid | (μL · min⁻¹) | (ppb) | (ppb) | (ppb) | (ppb) |
| H₂O | 2 | 32 | 55 | 29 | 83 |
| Polysiloxane- | 2 | 436 | 85 | 105 | 95 |
| EDTA | | | | | |
| Polysiloxane- | 2 | 702 | 56 | 78 | 84 |
| DTPA | | | | | |

### Example 11: Use of Polysiloxane-EDTA Nanoparticles as Perfusion Fluid for Cerebrospinal Fluid (CSF)

In order to model the CSF, a solution composed of NaCl (147 mM), KCl (2.7 mM), CaCl₂) (1.2 mM) and MgCl₂ (0.85 mM) was synthesized. The metal extraction was carried out with this solution in order to check that the extraction power was not diminished by the different ions that could interfere. A solution similar to the one in Example 9 was made up (i.e. 600 mL of reconstituted CSF containing 100 ppb of each of the ions Al (III), Cd (II), Zn (II), Cu (II) and Pb (II)). The microdialysis membrane (63 Microdialysis Catheter, M Dialysis AB, Sweden) used has a cut-off of 20 kDa and the flow rate was set at 2 μL/min-1 with a collection time of 40 min. The analysis of the extracted metal amounts was performed by ICP/MS. The perfusion fluid consisted of either reconstituted CSF or the polysiloxane-EDTA nanoparticles whose synthesis is described in Example 7 dispersed in the reconstituted CSF. The results of the extraction are given in Table 4. It can be noted that the perfusion fluid containing only the CSF has a very low extraction capacity. The addition of the nanoparticles to the perfusion fluid significantly increases the metal extraction regardless of the metal. In these conditions, a metal extraction factor of more than 5 for lead, more than 7 for copper, more than 25 for cadmium and more than 125 for aluminum is gained.

TABLE 4
Concentration of extracted metals in a CSF solution comparing
CSF and polysiloxane-EDTA nanoparticles (10 mM) as perfusion
fluid at a flow rate of 2 μL/min⁻¹.

| | Flow | | | | |
|---|---|---|---|---|---|
| CSF perfusion | rate | Al | Cu | Cd | Pb |
| fluid | (μL/min⁻¹) | (ppb) | (ppb) | (ppb) | (ppb) |
| CSF | 2 | 5 | 8 | 4 | 21 |
| Polysiloxane- | 2 | 643 | 63 | 112 | 116 |
| EDTA | | | | | |

1. Method for extracting metal cations from a biological fluid of a subject in need thereof, comprising dialyzing said subject with a dialysis device, said dialysis device comprising:
a porous dialysis membrane, and
a reservoir containing a perfusion fluid which comprises at least one chelator:
said at least one chelator being grafted on polymers, or
said at least one chelator being grafted on nanoparticles, wherein the cut-off threshold of the porous membrane is lower than the mass of the polymer or the nanoparticle to which the at least one chelator is grafted, and wherein said metal cations from a biological fluid of a subject are extracted by complexing to said chelator during dialysis.

2. Method according to claim 1 wherein said polymer is of more than 10 kDa.

3. Method according to claim 1 wherein said polymer is of more than 100 kDa.

4. Method according to claim 1 wherein said polymer is selected in the group consisting of the family of chitosans, polyacrylamides, polyamines or polycarboxylic acids.

5. Method according to claim 1 wherein said polymer is chitosan.

6. Method according to claim 1 wherein said nanoparticles are polysiloxane-based nanoparticles having a mean diameter greater than 3 nm.

7. Method according to claim 1 wherein said nanoparticles are polysiloxane-based nanoparticles having a mean diameter comprised between 3 nm and 50 nm.

8. Method according to claim 1 wherein said nanoparticles are polysiloxane-based nanoparticles having a mean size comprises between 20 kDa and 1MDa.

9. Method according to claim 1 wherein said chelators are in a proportion between 5 and 1000 per nanoparticle.

10. Method according to claim 1 wherein said the chelator is obtained by grafting onto the polymer or onto the nanoparticles, one of the following complexing molecules or its derivatives thereof: DOTA (1,4,7,10-tetraazacyclododecane-N, N′,N″,N′ “-tetraacetic acid), DTPA (diethylene triamine pentaacetic acid), DO3A-pyridine of formula (I), 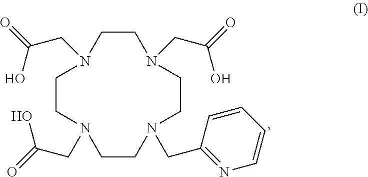 [SMILES: O=C(O)CN1CCN(CC(=O)O)CCN(Cc2ccccn2)CCN(CC(=O)O)CC1]
EDTA (2,2′,2″,2″-(ethane-1,2-diyldinitrilo)tetraacetic acid), EGTA (ethylene glycol-bis (2-aminoethylether)-N, N,N′,N′-tetraacetic acid), BAPTA (1,2-bis (o-aminophenoxy) ethane-N,N,N′,N′-tetraacetic acid), NOTA (1,4,7-triazacyclononane-1,4,7-triacetic acid), DOTAGA ((2-(4,7,10-tris (carboxymethyl)-1,4,7,10-tetraazacyclododecan-1-yl) pentanedioic acid), DFO (deferoxamine), amide derivatives such as for example DOTAM (1,4,7,10-tetrakis (carbamoylmethyl)-1,4,7,10 tetraazacyclododecane) or NOTAM (1,4,7-tetrakis (carbamoylmethyl)-1,4,7-triazacyclononane), as well as mixed carboxylic acid/amide derivatives, phosphonic derivatives such as DOTP (1,4,7,10-tetraazacyclododecane-1,4,7,10-tetrakis (methylene phosphonate)) or NOTP (1,4,7-tetrakis (methylene phosphonate)-1,4,7-triazacyclononane), cyclam derivatives Docket: 13500117US2 such as TETA (1,4,8,11-tetraazacyclotetradecane-N,N′,N″,M″-tetraacetic acid), TETAM (1,4,8,11-tctraazacyclotetradecane-N,N′,N″,M″-tetrakis (carbamoylmethyl)), TETP (1,4,8,11-tetraazacyclotetradecane—and N,N′,N″,N′ “-tetrakis (methylene phosphonate)) DTPABA (diethylenetriaminepentaacetic acid bianhydride) or mixtures thereof.

11. Method according to claim 1 wherein said the chelator is obtained by grafting onto the polymer or onto the nanoparticles DOTAGA ((2-(4,7,10-tris (carboxymethyl)-1,4,7,10-tetraazacyclododecan-1-yl) pentanedioic acid).

12. Method according to claim 1 wherein said metal cations are selected from cations of the metals Cu, Fe, Zn, Hg, Cd, Pb, Mn, Mg, Ca, Gd and Al.

13. Method according to claim 1 wherein said metal cations are selected from cations of the metals Cu and Fe.

14. Method according to claim 1 wherein said perfusion fluid contains trace elements selected from Calcium, Magnesium, Iron, Copper, Zinc and Manganese.

15. Method according to claim 1 wherein said biological fluid is blood.

16. Method according to claim 1 wherein said subject in need thereof, is suffering from a disease associated with metal overload or from a pathology related to a deregulation of metallic homeostasis.
